(19) United States
(12) Patent Application Publication
(10) Pub. No.: US 2010/0160214 A1
(43) Pub. Date: Jun. 24, 2010

(54) DESMOPRESSIN COMPOSITION

(75) Inventors: Seymour Fein, New Canaan, CT (US); Samuel Herschkowitz, Brooklyn, NY (US)

Correspondence Address: AXINN, VELTROP & HARKRIDER LLP;Attn. Michael A. Davitz, 114 West 47th Street, New York, NY, 10036, US

(73) Assignee: SERENITY PHARMACEUTICALS CORPORATION, Milford, PA (US)

(21) Appl. No.: 12/644,127

(22) Filed: Dec. 22, 2009

Related U.S. Application Data
(60) Provisional application No. 61/139,943, filed on Dec. 22, 2008.

(51) Int. Cl. A61K 38/12; A61P 7/12

(52) U.S. Cl. 514/11

(57) ABSTRACT

Disclosed is a pharmaceutical formulation that may be used in the treatment of nocturia, primary nocturnal enuresis, incontinence, urinary frequency, diabetes insipidus, or any disease or syndrome where desmopressin therapy is useful or where safe temporary suppression of urine production may lead to beneficial health effects or increased convenience in voiding control.

### FIELD OF THE INVENTION

The invention relates to compositions and devices for intra nasal administration of desmopressin so as to induce antidiuretic effects such as voiding postponement in a patient while minimizing the likelihood that the patient suffers from hyponatremia.

### BACKGROUND OF THE INVENTION

Desmopressin (1-desamino-8-D-arginine vasopressin, dDAVP®) is an analogue of vasopressin. Desmopressin has decreased vasopressor activity and increased anti-diuretic activity compared to vasopressin, and, unlike vasopressin, does not adversely effect blood pressure regulation. This enables desmopressin to be used clinically for anti-diuresis without causing significant increases in blood pressure. Desmopressin is commercially available as the acetate salt and is commonly prescribed for primary nocturnal enuresis (PNE) and central diabetes insipidus.

Desmopressin is a small peptide and is characterized by poor bioavailability. For treatment of severe illness such as cranial diabetes insipidus, it may be administered intravenously or subcutaneously, routes which essentially are 100% bioavailable. When taken in the commercialized dose forms of oral, sublingual and nasal spray delivery, bio availability is very low. Oral doses (pills) have a bioavailability far less than one percent, produce a wide range of blood concentrations of the drug depending on many factors, and produce a generally indeterminate duration of antidiuretic effect. Administration of desmopressin via the buccal mucosa and trans dermally also have been suggested. Intra nasal dosage forms have been approved for treatment of PNE, but the commercially available product (Minirin™) has now been declared to be unsafe for this use.

Hyponatremia is a condition in which the sodium concentration in the plasma is too low, e.g. below about 135 mmol/L. Severe hyponatremia can result in electrolyte abnormalities that can cause cardiac arrhythmias, heart attack, seizures or stroke. A hyponatremic state in patients administered desmopressin therapy occurs when the water channels in the kidneys of the patient are activated by the drug and the patient consumes aqueous liquids. This can but does not always result in lowering of blood osmolarity, lowering of sodium concentration, and consequent neurological damage. Some patients on a desmopressin regimen exhibit hyponatremia suddenly after having taken the drug without incident for long periods. Others develop the condition very early in the therapeutic regime. In short, the incidence of hyponatremia has largely been regarded as a stochastic side effect of the antidiuretic desmopressin therapy, avoidable only by avoidance of fluid intake while under the drug's effect.

Recent deaths from hyponatremia have been attributed to over intake of water while under the influence of desmopressin. As a result of these experiences, the U.S. Food & Drug Administration recently has warned physicians that use of desmopressin should be curtailed, that it is no longer indicated as appropriate for certain conditions, such as primary nocturnal enuresis (PNE), and has “Black Boxed” the drug. The recent warning stated that “[c]ertain patients, including children treated with the intranasal formulation of [desmopressin acetate] for primary nocturnal enuresis (PNE), are at risk for developing severe hyponatremia that can result in seizures or death.”

Currently, approved labeling for desmopressin administered intra nasally for treatment of PNE indicates bioavailability in the formulation is 3-5% and recommends dosing 10-40 micrograms per day. The average maximum plasma/serum concentrations achieved (Cₘₐₓ) with a typical intranasal dose (20 μg, 10 μg in each nostril) of desmopressin for PNE is at least approximately 20-30 pg/ml, based on 3-5% bioavailability with a 6 to 10 fold range. While existing formulations of desmopressin have proven to be adequate for many patients when used for these clinical indications, variable efficacy and occasional hyponatremic episodes continue to be problems related to the aforementioned variability.

U.S. Pat. No. 7,405,203 discloses antidiuretic therapy methods and desmopressin dosage forms. It discloses that the threshold plasma concentration for activation of the antidiuretic effect of desmopressin in humans is very low, less than about 1.0 pg/ml, and based in part on this observation, proposes the use and teaches how to make and use novel low dose desmopressin dosage forms that can substantially avoid the stochastic and unpredictable onset of hyponatremia. This is accomplished by administration of a very low dose of the drug, a dose sufficient to raise the desmopressin concentration in the blood only slightly above its threshold (e.g., about 0.5 pg/ml) from about 1.0, to about 10, and perhaps as high as 15 pg drug per ml of blood in some patients, but preferably no greater than about 10 pg/ml. This low concentration was discovered to be sufficient to induce potent antidiuretic effects of limited and controlled duration. Thus, the low blood concentration in combination with the known, approximate 90+ minute half life of desmopressin in a healthy person can function to control the “off switch” of the drug's activity and thereby to limit the duration of antidiuresis. This very significantly reduces the likelihood that the patient will drink sufficient liquids during the interval the drug is physiologically active such that the patient's homeostasis mechanisms are overwhelmed and blood sodium concentration falls to dangerous levels.

For example, in the treatment of nocturia (awakening from sleep to void at night) a low dose producing, e.g., a blood concentration of 5-7 pg/ml, can be administered at bed time. In less than about one half hour, desmopressin concentration is at its maximum of about 7 pg/ml, and urine production is suppressed. After two hours (one half life) the desmopressin concentration falls to about 3.5 pg/ml, at 3.5 hr (second half life), concentration is about 1.75, at 5 hr, approximately 0.85, and at 6 hours the concentration has fallen below the activation threshold (in many patients about 0.5 pg/ml) and the patient is making urine normally. If he retires at 11:00 PM, during the first six hours the patient makes little or no urine, his bladder is essentially empty, and his urge to urinate is accordingly suppressed. By 5 AM or so, urine production is restored and in an hour or two the patient wakes to urinate. As another example, a small dose, say, one sufficient to produce 2-3 pg/ml administered intra nasally or through a trans or intradermal patch, can induce safe antidiuresis for about three hours before normal urine production is restored.

Intra nasal administration is an attractive dosage route, and if one could formulate an intra nasal dosage form that would consistently produce a desmopressin blood concentration within or near the desired low dose range disclosed in the '203 patent, the incidence of the hyponatremia side effect would be reduced or eliminated, and the drug could be used safely as a convenience, as well as for the management of serious and bothersome conditions. While it clearly is within the skill of the art to produce a low dose intranasal desmopressin formulation that will be serviceable and induce safe antidiuresis reproducibly, the ideal intranasal dose form would, from one administration to the next, and from batch to batch, consistently produce a blood concentration within a relatively narrow target blood concentration range. It also would be desirable to formulate such a product so as to minimize the chances of abuse (multiple dosing) that could lead to antidiuresis of longer duration and potentially the development of hyponatremia. Because of variability in the human nasal mucosa, its permeability, the small amount of active peptide per dose, and many physical factors involved in self-administration of an intra nasal drug product, the product's bioavailability necessarily varies from person to person and use to use.

Thus, there is a need for a stable, easily administered desmopressin formulation that can be used to deliver low doses of desmopressin that is not associated with harmful side effects such as hyponatremia.

### SUMMARY OF THE INVENTION

In accordance with the invention, the properties of the spray composition enables respective doses of spray to be effective to restrict the concentration of desmopressin produced in the bloodstream of patients, on a per kilogram basis, to a relatively narrow range, thereby to achieve a relatively consistent, time limited duration of antidiuresis. Stated differently, respective successive spray doses establish in a patient by drug transport across intranasal mucosal membranes a Cₘₐₓ of desmopressin which is relatively consistent. The amount of drug delivered to the blood stream for repeated doses from the same dispenser to the same person preferably should differ no more than 100%, and preferably less than 50%. The dispenser's coefficient of variation is similar to the coefficient of variation of Cₘₐₓ produced by serial subcutaneous doses of desmopressin designed to achieve the same target Cₘₐₓ. Preferably, respective successive spray doses are sufficient to establish in a patient by intranasal delivery a Cₘₐₓ of desmopressin having a coefficient of variation within about 50%, more preferably about 25%, of the coefficient of variation of Cₘₐₓ produced by a subcutaneous dose of desmopressin designed to achieve the same target Cₘₐₓ. This consistency of bioavailability also is reflected in another property of dispensers of the invention, namely, they serve to establish in a patient by drug transport across intranasal mucosal membranes delivery of blood concentrations of desmopressin substantially directly proportional to the mass of desmopressin dispensed into the nostril(s) of a patient. This permits self titration of the length of antidiuresis desired by a patient.

The invention provides methods of inducing safe antidiuresis and pharmaceutical compositions in the form of an emulsified nasal spray comprising a Hsieh permeation enhancer having the following structure:

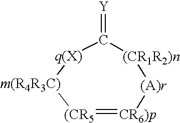

wherein X and Y are oxygen, sulfur or an imino group of the structure

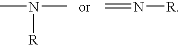

with the proviso that when Y is the imino group, X is an imino group, and when Y is sulfur, X is sulfur or an imino group, A is a group having the structure

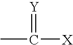

wherein X and Y are defined above, m and n are integers having a value from 1 to 20 and the sum of m+n is not greater than 25, p is an integer having a value of 0 or 1, q is an integer having a value of 0 or 1, r is an integer having a value of 0 or 1, and each of R, R₁, R₂, R₃, R₄, R₅ and R₆ is independently hydrogen or an alkyl group having from 1 to 6 carbon atoms which may be straight chained or branched provided that only one of R₁ to R₆ can be an alkyl group, with the proviso that when p, q and r have a value of 0 and Y is oxygen, m+n is at least 11, and with the further proviso that when X is an imino group, q is equal to 1, Y is oxygen, and p and r are 0, then m+n is at least 11.

The composition also comprises a liquid carrier, an emulsifying agent, and a therapeutically effective amount of desmopressin, such that when administered nasally, the pharmaceutical composition reliably achieves a target desmopressin Cₘₐₓ ranging from about 0.1 pg/ml to about 15+/−3 pg/ml, in many cases to about 10.0+/−3 pg/ml. The emulsifying agent may be a non-ionic surfactant.

In a preferred embodiment, the enhancer used in the composition is cyclopentadecalactone or cyclohexadecanone.

The Hsieh enhancer can be present in an amounts ranging from about 0.1% w/w to about 10% w/w, and in the currently preferred embodiment is about 2%. After nasal administration of the composition, the desmopressin Cₘₐₓ has a coefficient of variation within about 50% or less, preferably 25% or less of that produced by a subcutaneous dose of desmopressin designed to achieve about the same Cₘₐₓ. The desmopressin may be present in the pharmaceutical composition at a concentration ranging from about 1.0 μg/ml to about 50.0 μg/ml or from about 5.0 μg/ml to about 10.0 μg/ml, as the dose can be controlled by the quantity of composition delivered to the nasal mucosa per spray. The dose administered (applied to the nasal mucosa) can vary between 250 and 2500 ng of desmopressin. The dose delivered (the quantity that reaches the blood stream) can vary between 25 and 250 ng. After nasal administration of the pharmaceutical composition, the AUC_{0-∞} of desmopressin ranges from about 3.0 pg-hr/ml to about 20.0 pg-hr/ml, and the Tₘₐₓ of desompressin is achieved during a period ranging from about 0.25 hour to about 1.0 hour. The desmopressin Cₘₐₓ is directly proportional to the amount of nasally administered desmopressin over a Cₘₐₓ ranging from about 0.5 pg/ml to about 10.0 pg/ml. About 20 minutes after administration of the pharmaceutical composition of the present invention, the mean urine output per minute in a treated individual decreases to less than about 4 ml/minute, preferably less than about 1 ml/min, and stays in this range for a desired time period, such as 180 minutes, 240 minutes, 300 minutes, 360 minutes, or 420 minutes. About twenty minutes after administration, the mean urine osmolarity is greater than about 300 mOsmol/kg and remains at high concentration for a period of time ranging up to 180 minutes, 240 minutes, 300 minutes, 360 minutes, or 420 minutes.

The value of the target Cₘₐₓ may be varied, depending on the duration of the antidiuretic interval the dispensed composition is designed to induce. For example, a product designed for a 7-8 hour interval of urine production suppression might be designed to deliver a Cₘₐₓ of no more than 15+/−3 pg/ml. Thus, by way of illustration, a 7 hour product designed for children might have a bioavailability of 20% and a desmopressin load per spray of 0.75 μg or 750 ng. This would mean that about 150 ng of drug would reach the patient's blood stream, and that a 33 kg (˜75 lb.) child would achieve the target Cₘₐₓ of about 15 pg/ml. Another embodiment of the same product might have a bioavailability of 10% and a desmopressin load per spray of 1.5 μg or 1500 ng, again producing about 150 ng drug in the patient's bloodstream and the target Cₘₐₓ of about 15 pg/ml. Another exemplary product may be designed for a 3-4 hour urine interruption and might deliver a Cₘₐₓ of no more than about 3 pg/ml. Such a product, designed, for example, for use by women averaging 60 kg (˜130 lb.), might be 25% bioavailable and comprise a 250 ng desmopressin load per spray, or 15% bioavailable with a 350 ng load. In both cases, the bioavailable dose would be about 50 ng desmopressin, and the Cₘₐₓ about 3 pg/ml.

A primary and important property of the compositions of the invention is that they consistently deliver per spray a maximum blood concentration within a relatively narrow time and dose range, and therefore avoid or minimize accidental delivery of a larger dose resulting in a longer than expected antidiuretic effect and the possibility of induction of hyponatremia. Consistent delivery, as the phrase is used herein, should be taken to mean repeatable within a range similar to the range observed when administering very low doses of desmopressin by subcutaneous injection, or perhaps somewhat greater. Such consistency generally is achieved more easily exploiting formulations with higher bioavailability, and accordingly a bioavailability of at least 5%, preferably at least 10%, more preferably at least 15%, and preferably even higher is preferred. Higher bioavailability is achieved by exploiting formulation technology, especially the use of permeation enhancers as disclosed herein.

In various embodiments, the dispenser may be formulated to induce antidiuresis in a target patient population for less than six hours, for between 2 and 4 hours, or for between 4 and 7 hours. Maintaining the antidiuretic state for more than about 8 hours is not recommended. The target patient population may be, for example, children, children weighing less than 35 kg, children weighing between 35 and 50 kg, adult females, females weighing between 50 and 75 kg, adult males, males weighing between 70 and 85 kg, or males weighing more than 85 kg.

### BRIEF DESCRIPTION OF THE DRAWINGS

FIG. 1 is a graph of mean urine output vs. time (600 minutes) for men and women treated with 2000 ng intranasally administered desmopressin composition of the invention.

FIG. 2 is a graph of mean urine osmolarity vs. time for men and women treated with the same composition of the invention.

### DETAILED DESCRIPTION OF THE INVENTION

The term bioavailability is used to describe the fraction of an administered dose of drug that reaches the systemic circulation. By definition, when a medication is administered intravenously, its bioavailability is 100%. However, when administered via other routes, such as intranasally, bioavailability decreases due to incomplete absorption and other factors. Thus, bioavailability is a measurement of the extent of a therapeutically active drug that reaches the systemic circulation and is available at the site of action. It differs widely depending on chemical and physical properties of the drug in question and its route of administration. A quantity of the composition of the invention administered intra nasally refers to the quantity that exits the spray nozzle and enters the nostril(s). A quantity of the composition of the invention delivered refers to the quantity that actually reaches the bloodstream, i.e., becomes bioavailable. Proteins and peptides are relatively large and fragile molecules whose activity generally depends on their tertiary structure. The bioavailability of protein and peptide therapeutics administered other than parenterally is notoriously poor and variable.

The coefficient of variation, Cᵥ, as used herein, refers to a number expressed as a percentage that is a measure of the variability of the amount of and rapidity with which active drug gets into the blood stream when the same drug dose form is administered the same way, to the same person over many administrations or to many different persons. A coefficient of variation can be measured for Cₘₐₓ, Tₘₐₓ, or AUC. It is often expressed as the ratio of the standard deviation of a set of measurements to the mean of those measurements. Generally, intravenous or subcutaneous administration of any drug will have an inherently smaller Cᵥ as compared with trans dermal or oral administration. Intranasal administration of desmopressin is characterized not only by poor bioavailability, but also by a high Cᵥ. Thus, the commercially available Minirin® nasal spray product on the basis of Cₘₐₓ achieved per nasal spray dose has a high Cᵥ, 2 to 2.5 times that of subcutaneous injection. Thus, two patient's of the same weight using the same drug ostensibly the same way may experience widely varying blood concentrations of desmopressin, as measured, for example, using Cₘₐₓ, which may have a range of six to ten fold.

The coefficient of variation is calculated from measured blood concentrations. Accordingly, the imprecision of the analytical technique used to make the measurements comprising the raw data will contribute to Cᵥ. An assay with a large inherent error bar will produce a higher measured Cᵥ than an assay with a smaller error bar. When the measurements are made at the lower end of the dynamic range of an assay, where the standard deviation of the measurements is larger, Cᵥ as calculated based on the data will be larger than the Cᵥ of a larger dose of the same drug administered the same way and measured using the same assay.

The term “permeation enhancer,” as used herein, refers to one or a mixture of substances within the chemical genus described below which when formulated together with a peptide active, such as desmopressin, have the effect of increasing the fraction of the peptide applied to a nasal mucosal surface that traverses the mucosal membrane and enters the bloodstream, i.e., increases bioavailability. Generally, the addition of a permeation enhancer to a peptide drug formulation designed for intra nasal administration will increase the fraction of peptide that reaches the circulation by at least about 25%, preferably at least 50%, and most preferably at least about 100%.

The invention herein provides in part, improvements in desmopressin compositions adapted for administration via nasal spray characterized by delivering through the nasal mucosal surfaces and into the circulation of a more consistent as well as a lower desmopressin dose so as to induce a predetermined time-limited antidiuretic effect. The nasal spray drug product contains desmopressin and a Hsieh mucosal permeation enhancer which functions to promote passage of the peptide drug through the nasal mucosa. The active typically is dissolved or suspended in solutions or mixtures of excipients (e.g., preservatives, viscosity modifiers, emulsifiers, buffering agents, etc.) in a pressurized, but preferably non-pressurized, dispenser that delivers a specifically controlled amount of spray containing a metered dose into one or both nostrils. The dose typically is metered by the spray pump, which is typically finger or hand actuated. The nasal spray is designed for discharge of multiple spray doses, e.g., 10 to 100 or more. It may be designed to administer the intended dose with multiple sprays, e.g., two sprays, e.g., one in each nostril, or as a single spray, or to vary the dose in accordance with the weight, sex, or maturity of the patient, or to permit variation by the patient of the duration of antidiuresis.

One object of the design of the safety spray device is to assure to the extent possible that a consistent low concentration of desmopressin (the “target concentration”) is delivered to the bloodstream, e.g., generally not more that an amount sufficient to produce a maximum blood concentration of 15+/−3 pg/ml, and preferably less than 10 pg/ml. In many cases the device will deliver an amount of drug which achieves a blood concentration of 5+/−3 pg/ml or less.

The technical difficulty of achieving this goal is presented by the low and variable bioavailability of intranasally administered peptides, including desmopressin, by the very small amounts of active being administered, and by the low target blood concentrations. To promote consistent bioavailability, the concentration of active drug ingredient per spray and the mass (amount or load) of active per spray must be controlled to control precisely the amount of active that enters a nasal passage. This involves formulation of the drug and selection of design parameters of the pump spray using known methods. However, the amount of active that reaches the nasal mucosa can depend, upon other factors, on the physical composition of the spray, i.e., total amount injected, fluid properties such as viscosity, the momentum of the spray, and its droplet size distribution. These properties also are controlled by the chemistry of the formulation and spray nozzle characteristics. Overlaid on these factors determining bioavailability is that only a portion of the fraction of active reaching the mucosa successfully traverses this membrane and enters the blood stream. Unabsorbed drug is swallowed or otherwise degraded and is not bioavailable. Trans-mucosal passage of peptides is enhanced by including in the formulation certain substances that act as permeation enhancers. Of course, inconsistent spray procedure and the patient's particular nasal anatomy also play a part, but the inconsistency in drug uptake due to these factors cannot be controlled except by physician and/or packaging instructions for use that are explicit and clear and followed by the patient.

Applicants discovered that it is possible to safely administer desmopressin intra nasally by producing a composition exploiting these design principles as disclosed herein. The applicants have also discovered various desmopressin pharmaceutical formulations that may be administered safely with intra nasal dispensers.

A product designed, for example, to treat nocturia (urinary voiding at night interrupting sleep) in adults, to treat bed wetting in children (primary nocturnal enuresis), or to prevent bed wetting by a person suffering from incontinence, ideally would be taken by the patient after urinating at bedtime. Ideally the dose would suppress urine production for at least five hours, ideally six to six and a half, and possibly as much as eight. A product designed to interrupt urine production for a few hours during the day, such as to take a car trip for three or four hours, should interrupt urine production for two-three hours. At the end of the antidiuretic interval the healthy body seeks homeostasis rapidly and urine is produced normally. Thus, the urge to urinate returns in the next hour or next few hours. The products described herein of course also may be used, preferably under the care of a physician, for more serious disease such as central diabetes insipidus.

Of course, all of the times recited above are approximate, as the duration of antidiuresis achieved in a given person taking a given dose will have a certain inevitable variability. However, the intent and effect of the practice of the invention is to assure to the extent possible that a dose designed to last overnight does not in fact produce only three hours of antidiuresis, resulting in early waking, or involuntary voiding. More important, the effect of practice of the invention is to minimize the possibility that the interval of antidiuresis lasts unexpectedly long, e.g., 10 or 12 hours, resulting in an awake patient drinking liquids, and possibly developing hyponatremia.

The urine production suppression begins when the patient's desmopressin blood concentration exceeds the activation threshold of the water channels in the proximal kidney tubules, and ends when the concentration falls below that threshold. The exact concentration which is sufficient in a given individual to activate the water channels will vary, and it is so low that it is hard to measure with precision, but as disclosed in U.S. Pat. No. 7,405,203, experiments suggest the threshold is somewhat less than 1.0 pg/ml, or about 0.5 pg/ml, and possibly somewhat lower.

Table 1 illustrates certain important features of various embodiments of the invention. Referring to the Table, it discloses dosage parameters, ranges of maximum expected blood concentrations, the average weight of members of various patient populations, and expected durations of antidiuresis for each population. All listed dose forms are exemplary only and should not be regarded as limiting, except as otherwise indicated in the claims. All these products assume that one spray equals one dose. Of course multiple sprays could be employed to achieve the same dose and this may be desirable as promoting consistent uptake.

The first two products exemplify alternative ways to achieve antidiuresis for the treatment of nocturia in adult males. Both generate a Cₘₐₓ of about 5-8 pg/ml, but the first has a 10% bioavailability and delivers 1.0 to 1.6 μg desmopressin per spray, while the second has a bioavailability of about 20%, so requires only about half as much active per spray. Both deliver about 100 to 160 ng of drug to the patient's bloodstream, and this amount circulates to produce the desired blood concentration (Cₘₐₓ). Exemplary product 3 is designed to treat enuresis in children. If the child has an average weight of 35 kg, he or she will experience 5 to 7 hours of antidiuresis with an intranasal dose of 300-400 ng and a 15% bioavailability. This will deliver 45-70 ng desmopressin to the child's circulation and produce the desired 5-8 pg/ml concentration that will fall below the threshold concentration as normal clearance mechanisms reduce drug concentration until the threshold is passed five to seven hours later. Exemplary product 4 is designed to induce short duration urine suppression in, e.g., females averaging 60 kg. In this case, the interval desirably is short, e.g., about three hours. This can be achieved by intranasal administration of a dose that will produce a Cₘₐₓ of 1-2 pg/ml. This blood concentration can be achieved reliably with proper use of a dispenser delivering a 100-200 ng load characterized by a 15% bioavailability. Products 5 and 6 illustrate still other products designed for treatment of nocturia or other therapies involving temporary suppression of urine production in a 60 kg woman or a 200 kg man.

TABLE 1

| | | | | Mass of | | | | |
|---|---|---|---|---|---|---|---|---|
| | Patient | Duration of | | Drug per | | | Drug Delivered | |
| | Population | Antidiuresis | | Spray | | Bioavailability | to Bloodstream | Cmax |
| 1 | 70 kg | 5-7 | hr | 1.0-1.6 | μg- | 10% | 100-160 ng | 5-8 pg/ml |
| | adults | | | | | | | |
| 2 | 70 kg | 5-7 | hr | 500-800 | ng | 20% | 100-160 ng | 5-8 pg/ml |
| | adults | | | | | | | |
| 3 | 35 kg | 5-7 | hr | 300-480 | ng | 15% | 45-70 ng | 5-8 pg/ml |
| | children | | | | | | | |
| 4 | 60 kg adult | 3 | hr | 100-200 | ng | 15% | 15-35 ng | 1-2 pg/ml |
| | females | | | | | | | |
| 5 | 60 kg adult | 5-7 | hr | 400-700 | ng | 20% | 80-140 ng | 5-8 pg/ml |
| | females | | | | | | | |
| 6 | 100 kg | 5-7 | hr | 3-4.5 | μg | 5% | 140-220 ng | 5-8 pg/ml |
| | adult males | | | | | | | |

Turning now to the details of the dispenser, suitable drug reservoir's such as glass bottles and plastic squeeze bottles are widely available and used for pharmaceutical dispensing. Preferably the reservoir and the spray pump are disposable. Finger actuated pump sprays comprising plastic parts and metal springs are available commercially, for example, from Pfeiffer of America, Inc, Princeton N.J. These are available in designs to control drop size distribution to meet various specifications. For use in intranasal products the pumps typically deliver a 100 μl load in a narrow spray pattern, although in various embodiments of the invention the volume per spray may be varied, e.g., between 50 μl and 150 μl. Many different such metered drug pump designs can be adapted for use in the invention. Non limiting examples are disclosed in U.S. Pat. Nos. 4,860,738, 4,944,429, 6,321,942, 6,446,839, 6,705,493, 6,708,846, 6,772,915, and 7,182,226.

Each spray comprises a multiplicity of droplets, preferably with an average volume distribution in the range of 20 μm for D10 to about 300 μm for D90. This means that about 10% of the droplets are smaller than about 20 μm in diameter and 90% are smaller than 300 μm in diameter. Each spray dose is of a weight and desmopressin concentration such that it comprises between 0.5 ng desmopressin per kilogram of the patient's body weight and 75 ng desmopressin per kilogram of the patient's body weight. The spray is characterized by a desmopressin bioavailability greater than about 5%, that is, between about 5% and 25% of the active in the composition actually enters the patient's bloodstream and contributes to the drug effect, and the remainder is degraded, typically by digestion. Generally, the higher the bioavailability of a spray, the less desmopressin per spray needs to be delivered into a nasal cavity, and vice versa, the goal being to achieve more consistently a target desmopressin maximum blood concentration (Cₘₐₓ) in members of the patient population.

The currently preferred spray apparatus is sold as the Pfeiffer APF pump and is fitted to a 5.0 ml glass bottle. It delivers a metered, 100 μl load in a narrow spray pattern. Preferably, to promote consistency, the spray delivers the active formulation as a multiplicity of droplets with an average volume distribution in the range of 20 μm for D10 to about 300 μm for D90. This means that about 10% of the droplets are smaller than about 20 μm in diameter and 90% are smaller than 300 μm in diameter. Other distributions may be used. Very small droplets tend to be inhaled and may or may not reach the circulation. Large droplets may not penetrate the nostril lumen sufficiently and may result in leakage and loss. Such metered pumps assure that, with proper injection protocol, each use results in expelling a metered amount and that a relatively constant amount ends up in contact with the nasal mucosal surface.

The composition disposed within the reservoir comprises desmopressin, also called Anti-Diuretic Hormone, 1-desamino-8-D-arginine vasopressin, or dDAVP. It is a water soluble vasopressin analog having a molecular weight of 1069.23. Drug grade material is widely commercially available as the acetate salt. The term desmopressin, as used herein, refers to 1-desamino-8-D-arginine vasopressin and all other such analogs having antidiuretic activity, including analogs of active allelic variants of human vasopressin, and including other anions. See, for example U.S. Pat. Nos. 3,980,631 and 4,148,787.

The composition also necessarily includes at least one substance that acts as a permeation enhancer, that is, a substance which increases the net peptide transport across the mucosal membranes from the nasal lumen to the capillary bed behind it. Many potentially useful permeation enhancers are known in the art, and there are many ways to formulate such enhancers with peptide drugs so as to effectively increase their bioavailability. Permeation enhancers generally function by opening the tight junctions formed between epithelial cells of the mucosal membrane, thereby allowing diffusion of therapeutic agent into and through the membrane.

The permeation enhancer used in the composition of the invention are the so called Hsieh enhancers. See U.S. Pat. Nos. 5,023,252, 5,731,303, 7,112,561, and 7,244,703. The preferred Hsieh permeation enhancer having the following structure:

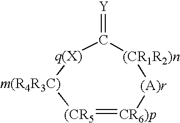

wherein X and Y are oxygen, sulfur or an imino group of the structure

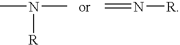

with the proviso that when Y is the imino group, X is an imino group, and when Y is sulfur, X is sulfur or an imino group, A is a group having the structure

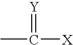

wherein X and Y are defined above, m and n are integers having a value from 1 to 20 and the sum of m+n is not greater than 25, p is an integer having a value of 0 or 1, q is an integer having a value of 0 or 1, r is an integer having a value of 0 or 1, and each of R, R₁, R₂, R₃, R₄, R₅ and R₆ is independently hydrogen or an alkyl group having from 1 to 6 carbon atoms which may be straight chained or branched provided that only one of R₁ to R₆ can be an alkyl group, with the proviso that when p, q and r have a value of 0 and Y is oxygen, m+n is at least 11, and with the further proviso that when X is an imino group, q is equal to 1, Y is oxygen, and p and r are 0, then m+n is at least 11. Cyclopentadecalactone or cyclohexadecanone are currently preferred, see U.S. Pat. No. 7,244,703. The currently preferred species is cyclopentadecanolide, sold under the trade name CPE-215 by CPEX, Inc of Exeter, N.H.

The enhancer is present in the composition in a concentration effective to enhance penetration of the pharmaceutically active peptide that is to be delivered through the nasal mucosa. Various considerations should be taken into account in determining the amount of enhancer to use. Such considerations include, for example, the amount of flux (rate of passage through the membrane) achieved and the stability and compatibility of the components in the formulations. The enhancer is generally used in an amount of about 0.1 to about 10 wt. % of the composition, and more generally in an amount of about 1.0 to about 3 wt. % of the composition.

The precise nature and amount of enhancer will vary depending on, for example, the particular permeation enhancer or enhancer composition selected, and on the nature of other components in the formulation. Thus, the concentration of the permeation enhancer within the medicament medium may be varied depending on the potency of the enhancer. The upper limit for enhancer concentration is set by toxic effect to or irritation limits of the mucosal membrane. The solubility of the enhancer within the medicament medium may also limit enhancer concentration.

The composition may be formulated as a simple, typically mildly acidic, aqueous solution of desmopressin, containing a water-soluble permeation enhancer molecule or multi-component permeation enhancer composition. Alternatively, the composition may be formulated as a two phase system with a hydrophobic and a hydrophilic phase. The composition of course may include other conventional components such as emulsifiers or surface active agents to aid in stabilization and enhancement of drop formation within the structure of the spray nozzle, preservatives so as to enhance shelf life or permit room temperature storage, stabilizers, osmolarity controls (salts), and a buffer or a buffer system. Formulations are best optimized empirically. Any given candidate formulation may be tested by intranasal administration to experimental animals, e.g., pigs or rats, or with proper approvals after appropriate pre clinical testing, to humans. Periodic sampling of blood will reveal the desmopressin concentration at various times post administration so as to permit calculation of Cₘₐₓ and other variables and the consistency of delivery to the circulation among successive doses both inter patient and intra patient.

The composition of the present invention may also comprise an emulsifying agent for use in aiding the formation of an emulsion. Essentially any suitable hydrocolloid emulsifying agent, or a mixture of two or more such emulsifying agents can be used in the practice of the present invention. Hydrocolloid emulsifying agents include: vegetable derivatives, for example, acacia, tragacanth, agar, pectin, and carrageenan; animal derivatives, for example, gelatin, lanolin, cholesterol, and lecithin; semi-synthetic agents, for example, methylcellulose and carboxymethylcellulose; and synthetic agents, for example, acrylic emulsifying agents such as carbomers. The hydrocolloid emulsifying agent forms hydrocolloids (hydrated lyophilic colloids) around the emulsified liquid droplets of the emulsion. The hydrocolloid serves as a protective layer around each emulsified droplet which physically repulses other droplets, thus hindering Ostwald ripening (the tendency of emulsified droplets to aggregate). In contrast, other emulsifying agents typically protect the emulsified droplets by forming a liquid crystalline layer around the emulsified droplets. In compositions which employ a liquid crystalline layer-forming emulsifying agent, the hydrophilic-lipophilic balance (HLB) of the oil phase of the emulsion must be matched with that of the emulsifying agent to form a stable emulsion and, often, one or more additional emulsifying agents (secondary emulsifying agents) must be added to further stabilize the emulsion. The aforementioned liquid crystalline layer also retards the release of the compounds of the dispersed phase upon contact with the target substrate.

The hydrocolloid emulsifying agents for use in the composition of the present invention include compounds which exhibit a low level of irritability or no irritability to the target membrane and which have good bioadhesive and mucoadhesive properties. Examples of hydrocolloid emulsifying agents which exhibit such properties include cellulosic emulsifying agents and acrylic emulsifying agents, including, for example, those which have an alkyl group containing from about 10 to about 50 carbon atoms. Particularly preferred acrylic emulsifying agents for use in the present invention are copolymers of a carboxylic acid and an acrylic ester (described, for example, in U.S. Pat. Nos. 3,915,921 and 4,509,949) with those which are cross-linked being especially preferred. An example of an emulsifying agent for use in forming an oil-in-water emulsion is “acrylates/C₁₀₋₃₀ alkyl acrylate crosspolymer”, a cross-linked polymer of acrylic acid and (C₁₀₋₃₀) alkyl acrylates. Acrylates/Cl₁₀₋₃₀ alkyl acrylate crosspolymer is available from Noveon, Inc. (previously B.F. Goodrich) and is sold under the trade name Pemulen™ Acrylates/C₁₀₋₃₀ alkyl acrylate crosspolymer has a small lipophilic portion and a large hydrophilic portion, thus allowing for it to function as a primary emulsifier for the formation of oil-in-water emulsions. In addition, acrylates/C₁₀₋₃₀ alkyl acrylate crosspolymer is capable of releasing the compounds of the dispersed phase upon contact with a substrate, namely, biological membranes or mucosa and will not re-wet (the oil phase will not re-emulsify upon contact with water). Additional information regarding acrylates/C₁₀₋₃₀ alkyl acrylate crosspolymer, which is listed in the U.S. Pharmacopeia, is provided in Noveon publications TDS-114, 117, 118, 124, 232-3, and 237, and PDS Pemulen 1622.

In forming an emulsion in which the water-insoluble enhancer is a normally solid material, the enhancer is dissolved in a suitable solvent. If the enhancer is a normally liquid material which is water-immiscible, a suitable solvent for the enhancer may or may not be used, as appropriate. The emulsifying agent is present in the composition in a concentration that is effective to form the desired liquid emulsion. In general the emulsifying agent is used in an amount of about 0.001 to about 5 wt. % of the composition, and more generally in an amount of about 0.01 to about 5 wt. % of the composition, and most generally in an amount of about 0.1 to about 2 wt. % of the composition.

The composition of the present invention may include, as an optional ingredient, particulate solids dispersed in the composition. For example, the composition may include an additional pharmaceutically-active compound dispersed in the liquid continuous phase of the emulsion in the form of microcrystalline solids or nanoparticulates.

### Example of Formulation Testing Protocol

This example describes how to test a given candidate formulation for efficiency in transport across nasal membranes. It assumes testing of compositions comprising water soluble permeation enhancers “A” and “B” and seeks to measure the fraction of desmopressin that permeates the nasal mucosa and enters the bloodstream in a low dose range, and how this bioavailability is altered as a function of the identity and concentration of these different enhancers.

Thus, by way of example, four formulations may be prepared having the following compositions.

Nasal formulation test compositions

| Formulation | 1 | 2 | 3 | 4 |
|---|---|---|---|---|
| Desmopressin | 2 | 2 | 2 | 2 |
| (μg/ml) | | | | |
| Na₂HPO₄ (mM) | 16 | 16 | 16 | 16 |
| Citric acid (mM) | 8 | 8 | 8 | 8 |
| NaCl (mM) | 145 | 145 | 145 | 145 |
| pH | 4.9 | 4.9 | 4.9 | 4.9 |
| Permeation | “A” | “A” | “B” | “B” |
| enhancer mg/ml | 2 mg/ml | 10 mg/ml | 2 mg/ml | 10 mg/ml |

A 10 μl drop of each formulation will contain 0.02 μg (20 ng) of desmopressin. A drop of a each candidate composition is applied to a nostril in each of three anesthetized rats, weighing, for example, between 225 an 250 grams. Blood is drawn prior to dosing and at 10, 20, 40, 60, and 120 minutes after dosing. The desmopressin concentration of each blood sample is determined using, for example, an immunoassay with sufficient sensitivity at the low pg desmopressin concentrations in the samples. From these data Cₘₐₓ can be calculated for each formulation and all compositions tested can be rated for efficient passage of desmopressin across rat nasal mucosal tissue. Promising formulations can be tested further, e.g., by introduction of a spray of a given formulation, volume and desmopressin concentration into the nostril of test pigs. Again, blood samples are drawn and Cₘₐₓ, AUC, or other measures of drug bioavailability can be determined. These data, in turn, permit preparation of test formulations for use in a phase I clinical trial, with the goal of designing a composition which when used correctly consistently produces a desmopressin drug concentration in the blood within a low dose target concentration range.

### Exemplary Formulation

Emulsion Stock Solution To produce an emulsion stock solution, the following ingredients in parts by weight are added to a vessel equipped with a stirring bar, and mixed for 15 minutes at 60-65° C.

180 parts sorbitan monolaurate (Span-20) aqueous solution (12 mg/ml)

30 parts Polysorbate 20 (Tween-20) aqueous solution (2 mg/ml)

400 parts cottonseed oil aqueous emulsion (26.6 mg/ml)

600 parts cyclopentadecanolide (CPE-215) aqueous emulsion (40 mg/ml)

Water to produce 1,500 grams total batch size

After mixing the preparation is homogenized using a high speed mixture at 6500 RPM+ for 20-25 minutes to produce a fine emulsion. This solution is autoclaved to assure sterility.

Buffer Solution To produce a citric acid buffer stock solution, the following ingredients in parts by weight are added to a vessel equipped with a stirring bar, and mixed for 5 minutes at 60-65° C.

6200 parts water

16 parts anhydrous citric acid aqueous solution (1.85 mg/ml)

76 parts sodium citrate, dihydrate aqueous solution (8.9 mg/ml)

104 parts Polysorbate 20 (Tween-20) aqueous solution (12 mg/ml)

Water to produce 8,500 grams total batch size

Desmopressin Solution To produce a desmopressin stock solution, 0.111 part desmopressin acetate trihydrate is added to sufficient buffer stock solution to produce 100.0 ml of solution, and stirred until all the desmopressin is dissolved to produce a stock solution having a concentration of 100 μg desmopressin/ml. From this stock solution a 10 μg/ml solution was prepared by dilution.

Aliquots of the 10 μg/ml solution were filtered to eliminate any bacterial contamination and diluted with an equal volume of emulsion stock solution to produce aseptic, preservative free dose forms comprising 5 μg/ml desmopressin, pH 5.5, containing 2% cyclopentadecanolide. These were bottled in sterile pump spray bottles fitted with a Pfeiffer APF pump sprayers that deliver 100 μl per metered spray, or 0.50 μg desmopressin, or 500 ng desmopressin per spray. The liquid contains no detectable microorganisms. The commercially available, disposable Pfeiffer APF pump comprises a mechanism that prevents back fill of potentially contaminated air after the pump has been actuated and thus maintains substantial sterility of each dose dispensed. These were tested on humans to determine the blood concentration they delivered, duration of antidiuresis, their pharmacokinetic properties, etc., as set forth below.

### Clinical Testing of Prototype Product

A clinical study using a safety dispenser embodying the invention described above in human adult subjects in a water loaded state demonstrated that doses administered intranasally of 500 ng to 2000 ng (one to four sprays) produced antidiuretic effects in a dose proportional relationship for durations of from 2 to 7 hours. Peak blood concentrations ranged from about 1.25 to about 10 pg/ml. None of the test subjects exhibited any drug related decreases in serum sodium.

The open-label preliminary study of the effects and pharmacokinetics of the prototype composition was conducted with six male and six female healthy, water loaded, non-smoking volunteer subjects, following the protocol described generally below. In summary, each subject was dosed up to four times over a period of one week with dosing administered every other day. On days one, three, and five subjects were dosed intranasally with escalating doses of the low dose desmopressin nasal spray formulation described above. On day seven, subjects were given a single bolus injection of low dose desmopressin either intradermally or subcutaneously as a comparison. All subjects were screened prior to the first treatment, including evaluations of medical history, complete physical exam including nasopharyngeal exam, serum chemistry including serum osmolality, urinalysis including urine osmolality.

On day one all subjects were asked to have his/her morning void prior to breakfast, and thereafter subjects started the water loading process. Water loading assures that a patient is not generating endogenous vasopressin, and accordingly permits isolation of the effect of exogenous desmopressin. To achieve a steady state diuresis, the subjects were directed to drink a volume of water corresponding to at least 1.5% and up to 3% of body weight. The water loading process started about two hours prior to the dosing of the first subject. Subjects were asked to void every 20 minutes. To ensure continuous water loaded state, the subjects replaced their urinary output loss with an equivalent amount of fluid. Insensible loss was not measured or replaced. When the urinary output rate exceeded 10 ml/min in two consecutive measurements (defined as water loaded state) in the subjects, dosing began. Subjects were maintained in the water loaded state with equivalent fluid intake versus fluid loss.

Each subject then was dosed intranasally with one 100 μl spray containing 0.5 μg (500 ng) of desmopressin nasal spray formulation in the right or left nostril. Urine volume was measured in 20-minute intervals from the start of water loading (at least two hours prior to dosing) to the time the subject's urine output returns to baseline (urinary output level that exceeds 10 ml/minute in three consecutive 20-minute measurements) post dose. Serum osmolality and sodium were measured prior to dosing and at 2, 4, 6 and 8 hours post dose.

Blood sampling for pharmacokinetic determinations was performed at 1, 1.5, 2, 3, 4 and 6 hours post dose. Two seven ml blood samples were collected at each time point. The concentration of desmopressin was determined by a validated radio immunoassay. The concentration of desmopressin in plasma was analyzed for the individual volunteer in each group, by use of non-compartmental methods using the commercially available software WinNonlin™Pro, ver. 3.2 (Pharsight Corporation, USA). A plasma concentration value below the limit of quantitation (“LOQ”) followed by values above the limit was set at ‘LOQ/2’ for the analysis and for the descriptive statistics on concentrations. Values below LOQ not followed by values above the LOQ were excluded from the analysis, and set to zero in the descriptive statistics on concentrations.

On days two, four, and six the subjects fasted beginning at 8 pm until breakfast the following day and were encouraged to drink one to two liters of water between 7 pm and 9 pm. Thereafter, they were to drink fluid ad libitum until the start of the water loading on the next day.

On day three, the subjects received one spray of desmopressin nasal spray formulation in each nostril (total volume of 200 μl equivalent to 1000 ng of desmopressin). Other than the dose level, all procedures were the same as those described for day one.

On day five, all subjects received a total volume of 2000 ng of desmopressin (one nasal spray in each nostril followed five minutes later by a second spray in each nostril). Other than the dose level, all procedures were the same as those described for day one.

On day seven, three male and three female subjects received a single bolus intradermal injection of desmopressin solution (150 μl of 0.8 μg/ml solution equivalent to 120 ng of desmopressin), and the other six subjects received a single bolus subcutaneous injection of desmopressin (150 μl of 0.8 μg/ml solution equivalent to 120 ng of desmopressin). Other than the dosing paradigm, all procedures were the same as described on day one.

Pharmacokinetic parameters were derived from the individual concentration of desmopressin found in blood samples versus time curves of desmopressin, included AUC, and Cₘₐₓ Assay values below the limit of detection of the desmopressin immunoassay (<1.25 pg/ml) were set equal to zero for purposes of averaging concentrations. Assay values below the level of detection that occurred between two non-zero concentrations were considered to be “missing” for purposes of calculating the AUC. Blood concentration measurements from the 0.5 μg dose study were not conducted as often unreliable and below the limit of detection. Since the traditional analysis resulted in many subject/treatment combinations not being evaluable for T_{1/2} or AUC, a hypothesis was made that for a given subject, the half-life would be consistent from treatment to treatment. Therefore, as long as one of the three treatments generated an evaluable terminal half-life, that value could be used to extrapolate the AUC for the treatments that did not have evaluable half-lives. Accordingly, an average terminal half-life (Avg T_{1/2}) was calculated for each subject that included a treatment with evaluable half-lives in that subject. Ten of the twelve subjects had half-lives evaluable for at least one treatment. The AUC could be calculated for each treatment and subject using the calculated average T_{1/2} value.

It was determined that aside from one anomalous patient, all 11 patients in the study had peak desmopressin drug concentrations at the 2000 ng dose level of between 3.9 and 10 pg/ml. Furthermore, 9 of the 11 achieved drug concentrations between 5.18 and 8.4 pg/ml. This alone illustrates the consistency of the blood concentration achieved using the prototype composition described above. Furthermore, as a result of the study, the following pharmacokinetic values were calculated. The calculated coefficient of variation of each data point is indicated in parentheses.

Results of Measured Pharmacokinetic Parameters (Mean ± SD (CV %))

| | | | 120 ng | 120 ng |
|---|---|---|---|---|
| | 1000 ng Nasally | 2000 ng Nasally | Subcutaneous | Intradermal |
| | (2 sprays) | (4 sprays) | Injection | Injection |
| Cmax | N = 7 | N = 12 | N = 6 | N = 6 |
| pg/mL | 2.79 ± 1.44 | 6.24 ± 2.25 | 2.77 ± 0.98 | 1.93 ± 0.46 |
| | (51.6%) | (36.0%) | (35.4%) | (23.8%) |
| Tmax | N = 7 | N = 12 | N = 6 | N = 6 |
| hr | 1.08 ± 1.32 | 0.35 ± 0.188 | 0.88 ± 0.349 | 0.80 ± 0.406 |
| | (122.7%) | (53.2%) | (39.5%) | (50.8%) |
| AUCₐₗₗ | N = 12 | N = 12 | N = 6 | N = 6 |
| pg · hr/mL | 2.43 ± 3.70 | 9.36 ± 6.67 | 4.01 ± 2.83 | 2.08 ± 1.89 |
| | (152.3%) | (71.3%) | (70.6%) | (90.8%) |
| AUC_{tlqc} | N = 12 | N = 12 | N = 6 | N = 6 |
| pg · hr/mL | 2.42 ± 3.44 | 9.16 ± 6.97 | 3.40 ± 2.63 | 1.87 ± 1.88 |
| | (141.9%) | (76.1%) | (77.3%) | (100.3%) |
| AUC∞ | N = 3 | N = 8 | N = 3 | N = 2 |
| pg · hr/mL | 6.49 ± 3.59 | 11.50 ± 7.9 | 10.22 ± 4.9 | 6.42 ± 3.83 |
| | (55.3%) | (68.6%) | (47.5%) | (59.6%) |
| AUC∞* | N = 10 | N = 10 | N = 6 | N = 4 |
| pg · hr/mL | 5.36 ± 5.92 | 11.59 ± 7.9 | 7.85 ± 4.21 | 4.46 ± 3.09 |
| | (110.5%) | (68.0%) | (53.6%) | (69.4%) |
| λz | N = 3 | N = 8 | N = 3 | N = 2 |
| 1/hr | 0.646 ± 0.198 | 0.639 ± 0.348 | 0.337 ± 0.048 | 0.549 ± 0.239 |
| | (30.6%) | (54.4%) | (14.4%) | (43.5%) |
| T_{1/2} hr | N = 3 | N = 8 | N = 3 | N = 2 |
| | 1.13 ± 0.30 | 1.33 ± 0.56 | 2.09 ± 0.32 | 1.39 ± 0.61 |
| | (26.3%) | (42.3%) | (15.4%) | (43.5%) |
| Avg T_{1/2} hr | N = 10 | N = 10 | N = 6 | N = 4 |
| | 1.49 ± 0.60 | 1.49 ± 0.60 | 1.68 ± 0.66 | 1.20 ± 0.42 |
| | (40.3%) | (40.3%) | (39.1%) | (34.8%) |
| CL/FL/hr | N = 3 | N = 8 | N = 3 | N = 2 |
| | 184 ± 83 | 232 ± 118 | 14 ± 6.0 | 23 ± 13.5 |
| | (45.0%) | (51.0%) | (43.9%) | (59.6%) |
| F % | N = 1 | N = 4 | N = 3 | N = 2 |
| | 18.8% ± NE | 7.4% ± 2.3% | 100.0% ± 0.0% | 55.0% ± 0.0% |
| | (NE) | (31.4%) | (0.0%) | (0.0%) |
| Extrapolated | N = 3 | N = 8 | N = 3 | N = 2 |
| AUC % | 47.4% ± 10.6% | 30.3% ± 14.2% | 56.0% ± 9.4% | 47.1% ± 10.8% |
| | (22.4%) | (46.9%) | (16.7%) | (22.8%) |

Assay values below the level of detection (<1.25 pg/ml) that occurred between two non-zero concentrations were considered to be “missing” for purposes of calculating the AUC_{tlqc}, AUC∞, AUC∞* and the terminal rate constant. Only results of the 1 and 2 μg dose studies are listed in the table above, as some of the pharmacokinetic results for the 0.5 μg dose study were below the limit of detection. However, those measurable results were proportional to the similar results observed for the 1 and 2 μg dose studies. Thus, the pharmacokinetics of desmopressin appear to be linear over the administered doses described in the study.

The AUCₐₗₗ is the AUC calculated using the trapezoidal rule that assumes that the concentration declines to 0.00 at the next sample collection time following the last quantifiable concentration. In the cases where concentrations had not declined to below the limit of detection by the time of the last scheduled sample, AUCₐₗₗ was the same as AUC_{tlqc}.

The AUC_{tlqc} is the AUC as calculated to the Time of the Last Quantifiable Concentration. This value is extrapolated using the ratio of the last quantifiable concentration and the terminal elimination rate to give the AUC∞. Therefore AUC∞ is always larger than the AUC_{tlqc}. AUCₐₗₗ will also always be equal to or greater than AUC_{tlqc}, but may be larger or smaller than AUC∞. As noted in the table the average fraction of the AUC that was estimated to be in the extrapolated portion was typically in the 50% range, although a bit lower (30% on average) after the highest nasal spray dose. λz and T_{1/2} were estimated using the terminal data points that formed an approximately straight line when plotted as ln(conc.) vs. time. At least 3 points had to be present in this segment, and the correlation coefficient for the slope equal to or greater than 0.80 for the terminal rate constant and half-life to be considered evaluable. Additional data points were sequentially added into the regression analysis as long as they improved the correlation coefficient, and they did not occur before Tmax.

Since the traditional analysis resulted in many subject/treatment combinations not being evaluable for T_{1/2} or AUC∞, a hypothesis was made that for a given subject, the half-life would be consistent from treatment to treatment. Therefore, as long as one of the three treatments generated an evaluable terminal half-life, that value could be used to extrapolate the AUC for the treatments that did not have evaluable half-lives. Therefore, an average terminal half-life (Avg T_{1/2}) was calculated for each subject that included all the treatments with evaluable half-lives in that subject. 10/12 subjects had half-lives evaluable for at least 1 treatment. A modified AUC∞ (AUC∞*) was calculated for each treatment and subject using that average T_{1/2} value. Those “special” AUC∞* are included in the mean table above.

The fraction bioavailable (F) was calculated as the ratios of the AUC∞ of the nasal treatment vs. either the SC or ID treatment, after adjustment for the differences in dose. For subjects where the reference treatment was the ID dose, the ratio of the dose corrected AUCs was further corrected by the average bioavailability of the ID dose compared to the SC dose (˜55%).

Two conclusions may be derived from these data. First, the coefficient of variation of Cₘₐₓ of desmopressin administered intranasally using the composition of the invention for the 1000 ng dose (51.6%) is only about 30% greater than coefficient of variation of Cₘₐₓ of a dose of desmopressin administered subcutaneously and designed to produce comparable low blood concentrations. The measured coefficient of variation of Cₘₐₓ of desmopressin administered intranasally using the dispensed composition of the invention for the 2000 ng dose (36.0%) is about equal to the coefficient of variation of Cₘₐₓ of the subcutaneous dose. These preliminary data support the hypothesis that the formulation of the invention indeed is characterized by a coefficient of variation of Cₘₐₓ comparable to that of subcutaneous desmopressin doses designed to achieve a comparable low blood concentration. This is in sharp contrast to commercially available intranasal desmopressin dose forms which, despite being designed to deliver far higher blood concentrations, have a much higher variation in Cₘₐₓ, a variation that contributes to the stochastic induction of a hyponatremic state.

Second, note that both AUC and Cₘₐₓ produced by this formulation dispensed intranasally appear to be directly linearly proportional to dose. Thus, the 1000 ng intranasal dose yields a Cₘₐₓ of 2.79+/−1.44 pg/ml, while the 2000 ng dose yields a value of 6.24+/−2.25; the 1000 ng intranasal dose results in an AUC of 5.36+/−5.92, which is approximately doubled to 11.50+/−7.9 when the dose is doubled. This suggests that desmopressin can be reliably dispensed intranasally to reproducibly achieve an antidiuretic effect of limited duration without substantial risk of members of a patient population developing hyponatremia. It also suggests that a dispenser delivering a low dose may be used via multiple sprays to achieve any of several antidiuresis durations in a given patient, or that one dispenser may be sold to service different patient populations provided there is proper instruction for how many sprays should be used to produce a given duration of effect in a given population.

The results of this study suggest that the low-dose desmopressin nasal spray embodying the invention provides improved, more reproducible pharmacokinetic parameters at relatively consistent low blood concentrations, and delivers a Cₘₐₓ proportional to the doses administered.

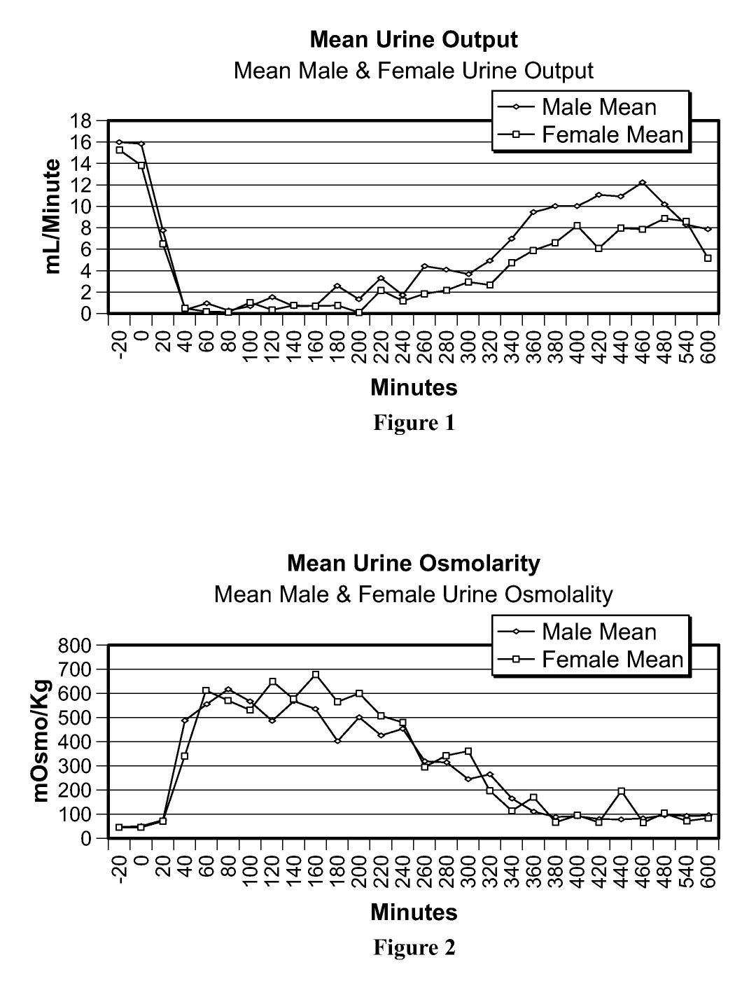

The urine output and urine osmolarity was measured just prior to nasal administration of 2000 ng of the pharmaceutical composition of desmopressin and for a period of up to about 10 hours (600 minutes) after administration. FIG. 1 shows the mean urine output for male and female subjects. As evidenced by the data, the urine output fell to less than 8 ml/minute within 20 minutes after administration of the desmopressin by nose (in water loaded individuals). Urine output remained less than 8 ml/minute for a period ranging up to about 400 minutes after administration. FIG. 2 shows the mean urine osmolarity for the same group of male and female subjects as in FIG. 1. Urine osmolarity increased to greater than about 400 mOsmol/kg within 40 minutes after administration of 2 μg of desmopressin nasally and remained greater than about 400 mOsmol/Kg for about 250 minutes after administration of the desmopressin by nose.

A second separate study in adult patients with nocturia established that doses of 500 and 1000 ng (one or two sprays administered intranasally) produced dramatic therapeutic decreases in the number of night time urinary voids equal to or less than one per night in 41 of 43 patients. Serum sodium levels remained within normal limits throughout treatment.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

Other embodiments are within the following claims.

1. A pharmaceutical composition in the form of an emulsified nasal spray comprising: a Hsieh permeation enhancer having the following structure: 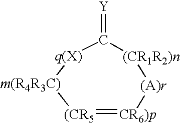
wherein X and Y are oxygen, sulfur or an imino group of the structure 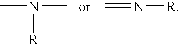
with the proviso that when Y is the imino group, X is an imino group, and when Y is sulfur, X is sulfur or an imino group, A is a group having the structure 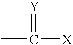
wherein X and Y are defined above, m and n are integers having a value from 1 to 20 and the sum of m+n is not greater than 25, p is an integer having a value of 0 or 1, q is an integer having a value of 0 or 1, r is an integer having a value of 0 or 1, and each of R, R₁, R₂, R₃, R₄, R₅ and R₆ is independently hydrogen or an alkyl group having from 1 to 6 carbon atoms which may be straight chained or branched provided that only one of R₁ to R₆ can be an alkyl group, with the proviso that when p, q and r have a value of 0 and Y is oxygen, m+n is at least 11, and with the further proviso that when X is an imino group, q is equal to 1, Y is oxygen, and p and r are 0, then m+n is at least 11,
a liquid carrier,
an emulsifying agent, and
a therapeutically effective amount of desmopressin, such that when a directed spray or number of sprays of the pharmaceutical composition is administered nasally to a human patient, a desmopressin Cₘₐₓ is produced in the bloodstream of the patient ranging from about 1 pg/ml to no more than about 15.0+/−3 pg/ml.

2. The composition of claim 1, wherein the Hsieh enhancer is cyclopentadecalactone or cyclohexadecanone.

3. The composition of claim 1, wherein the Hsieh enhancer is present in an amount ranging from about 0.1% w/w to about 10% w/w.

4. The composition of claim 1, wherein the desmopressin Cₘₐₓ has a coefficient of variation within about 50% or less of that produced by a subcutaneous dose of desmopressin designed to achieve about the same Cₘₐₓ.

5. The composition of claim 1, wherein the desmopressin Cₘₐₓ has a coefficient of variation within about 25% or less of that produced by a subcutaneous dose of desmopressin designed to achieve about the same Cₘₐₓ.

6. The composition of claim 1, wherein the desmopressin is present in the pharmaceutical composition at a concentration ranging from about 0.5 μg/ml to about 50.0 μg/ml.

7. The composition of claim 6, wherein the desmopressin is present in the pharmaceutical composition at a concentration ranging from about 5.0 μg/ml to about 10.0 μg/ml.

8. The composition of claim 1 wherein the AUC_{0-∞} of desmopressin after nasal administration ranges from about 3.0 pg-hr/ml to about 20.0 pg-hr/ml.

9. The composition of claim 1, wherein the Tₘₐₓ of desmopressin is achieved during a period ranging from about 0.25 hours to about 3.0.

10. The composition of claim 1, wherein the desmopressin Cₘₐₓ is directly proportional to the amount of nasally administered desmopressin over a Cₘₐₓ ranging from about 1 pg/ml to about 10.0 pg/ml.

11. The composition of claim 10, wherein the desmopressin Cₘₐₓ is directly proportional to the amount of nasally administered desmopressin over a Cₘₐₓ ranging from about 1.0 pg/ml to about 8.0 pg/ml and wherein nasally administered desmopressin ranges from about 250 ng to about 2500 ng.

12. The composition of claim 1, wherein the emulsifying agent is a non-ionic surfactant.

13. The composition of claim 1 characterized in that the patient's mean urine output per minute decreases to less than about 4 ml/minute about 20 minutes after the pharmaceutical composition is administered.

14. The composition of claim 1 characterized in that the patient's mean urine output per minute decreases to less than about 4 ml/minute for a period of time ranging up to about 180 minutes, 240 minutes, 300 minutes, 360 minutes, or 420 minutes.

15. The composition of claim 13 or 14 wherein the mean urine output is less than about 1 ml/minute.

16. The composition of claim 1 characterized in that the patient's mean urine osmolarity is greater than about 300 mOsmol/kg after about 20 minutes after the pharmaceutical composition is administered and remains above said concentration for a period of time ranging up to about 180 minutes, 240 minutes, 300 minutes, 360 minutes, or 420 minutes.

17. A method of inducing an antidiuretic effect by administering a pharmaceutical composition in the form of an emulsified nasal spray comprising: a Hsieh permeation having the following structure: 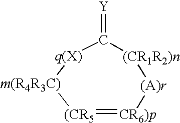
wherein X and Y are oxygen, sulfur or an imino group of the structure 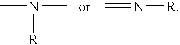
with the proviso that when Y is the imino group, X is an imino group, and when Y is sulfur, X is sulfur or an imino group, A is a group having the structure 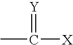
wherein X and Y are defined above, m and n are integers having a value from 1 to 20 and the sum of m+n is not greater than 25, p is an integer having a value of 0 or 1, q is an integer having a value of 0 or 1, r is an integer having a value of 0 or 1, and each of R, R₁, R₂, R₃, R₄, R₅ and R₆ is independently hydrogen or an alkyl group having from 1 to 6 carbon atoms which may be straight chained or branched provided that only one of R₁ to R₆ can be an alkyl group, with the proviso that when p, q and r have a value of 0 and Y is oxygen, m+n is at least 11, and with the further proviso that when X is an imino group, q is equal to 1, Y is oxygen, and p and r are 0, then m+n is at least 11,
a liquid carrier comprising water, an emulsifying agent, and
a therapeutically effective amount of desmopressin, such that when a directed spray or number of sprays of the pharmaceutical composition is administered nasally to a human patient, a desmopressin Cₘₐₓ is produced in the bloodstream of the patient ranging from about 1 pg/ml to no more than about 15.0+/−3 pg/ml.

18. The method of claim 17, wherein the Hsieh enhancer is cyclopentadecalactone or cyclohexadecanone.

19. The method of claim 18, wherein the Hsieh enhancer is present in an amount of about 2% of the composition.
